(19) United States
(12) Patent Application Publication
(10) Pub. No.: US 2022/0362016 A1
(43) Pub. Date: Nov. 17, 2022

(54) DILATION DEVICES, SYSTEMS, AND METHODS FOR IMPLANTS

(71) Applicant: Edwards Lifesciences Corporation, Irvine, CA (US)

(72) Inventor: Eran Goldberg, Nesher (IL)

(21) Appl. No.: 17/816,955

(22) Filed: Aug. 2, 2022

Related U.S. Application Data
(63) Continuation of application No. PCT/US2021/016736, filed on Feb. 5, 2021.
(60) Provisional application No. 62/971,086, filed on Feb. 6, 2020.

(51) Int. Cl. A61F 2/24; A61M 29/02

(52) U.S. Cl. CPC A61F 2/2433; A61M 29/02; A61M 25/1002

(57) ABSTRACT

Devices, systems, and methods may be used for dilating implants utilizing dilation devices. An implant deployment system may include an inflatable body having a central body configured to press an inner surface of the implant to dilate the implant and having a profile that decreases in diameter along a length of the central body. An inflatable body may include a plurality of segments with varying expansion characteristics.

### CROSS-REFERENCE TO RELATED APPLICATIONS

This application is a continuation of PCT Patent Application No. PCT/US2021/016736 filed Feb. 5, 2021, which application claims the benefit of and priority to U.S. Provisional Application No. 62/971,086 filed Feb. 6, 2020, each of these applications being incorporated herein by reference in its entirety.

### TECHNICAL FIELD

The present disclosure describes systems, devices, and methods related to implants, and devices, systems and methods of dilation.

### BACKGROUND

A variety of maladies may affect an individual's body. Such maladies may be of the individual's heart, and may include maladies of the individual's heart valves, including the aortic, mitral, tricuspid, and pulmonary valves. Stenosis, for example, is a common and serious valve disease that may affect the operation of the heart valves and an individual's overall well-being.

Implants may be provided that may replace or repair portions of a patient's heart. Prosthetic implants, such as prosthetic heart valves, may be provided to replace a portion of a patient's heart. Prosthetic aortic, mitral, tricuspid, and even pulmonary valves may be provided.

Implants may be deployed to the desired portion of the patient's body percutaneously, in a minimally invasive manner. Such deployment may occur transcatheter, in which a catheter may be deployed through the vasculature of an individual.

During deployment of such implants, the implants must be dilated to provide an expanded configuration for such implant. Care must be taken to property dilate the implants to avoid over expansion or under expansion of such implants and to properly deploy such an implant.

### SUMMARY

The present systems and methods may relate to devices, systems, and methods for dilating implants utilizing dilation devices. Such devices, systems, and methods may include inflatable bodies configured to be inflated to dilate the implant. In various embodiments, a dilation device for an implant may be provided. The dilation device may include an inflatable body having a first end and a second end and a central body positioned between the first end and the second end and having a length. The central body may be configured to press an inner surface of the implant to dilate the implant and may have a profile that decreases in diameter along the length of the central body from the first end to the second end.

In various embodiments, a dilation device for an implant may be provided. The dilation device may include an inflatable body having a first end and a second end and a central body positioned between the first end and the second end and configured to press an inner surface of the implant to dilate the implant. The central body may include a plurality of segments each having a different profile than each other of the plurality of segments. A plurality of imaging markers may each be configured to indicate a position of a respective one of the plurality of segments.

In various embodiments, an implant deployment system may be provided. The implant deployment system may include an implant configured to be deployed to a deployment location within a subject and having an angled interior profile that faces an interior cavity of the implant. The implant deployment system may include an inflatable body having a central body configured to be positioned within the interior cavity of the implant and configured to press towards the implant to dilate the implant, the central body having a profile that is angled to fit the angled interior profile of the implant.

In various embodiments, an implant deployment system may be provided. The implant deployment system may include a deployment apparatus for deploying an implant and having an elongate shaft. The implant deployment system may include an inflatable body coupled to the elongate shaft and having a first end and a second end and a central body positioned between the first end and the second end and having a length, the central body configured to press an inner surface of the implant to dilate the implant and having a profile that decreases in diameter along the length of the central body from the first end to the second end.

In various embodiments, an implant deployment system may be provided. The implant deployment system may include a deployment apparatus for deploying an implant and having an elongate shaft. The implant deployment system may include an inflatable body coupled to the elongate shaft and having a first end and a second end and a central body positioned between the first end and the second end and configured to press an inner surface of the implant to dilate the implant, the central body including a plurality of segments each having a different profile than each other of the plurality of segments. The implant deployment system may include a plurality of imaging markers coupled to the elongate shaft and each configured to indicate a position of a respective one of the plurality of segments.

In various embodiments, a method may be provided. The method may include dilating an implant utilizing an inflatable body having a central body pressed towards an inner surface of the implant, the inflatable body having a first end and a second end and a central body positioned between the first end and the second end, the central body having a length and having a profile that decreases in diameter along the length of the central body from the first end to the second end.

In various embodiments, a method may be provided. The method may include dilating an implant utilizing an inflatable body having a central body pressed towards an inner surface of the implant, the inflatable body having a first end and a second end and a central body positioned between the first end and the second end, the central body including a plurality of segments each having a different profile than each other of the plurality of segments.

The method may include aligning the implant with at least one of a plurality of imaging markers each configured to indicate a position of a respective one of the plurality of segments.

In various embodiments described herein, systems, devices and methods may be deployed or performed within a subject. Subjects include (but are not limited to) medical patients, veterinary patients, animal models, cadavers, and simulators of the cardiac and vasculature system (e.g., anthropomorphic phantoms and explant tissue).

### BRIEF DESCRIPTION OF THE DRAWINGS

These and other features, aspects, and advantages are described below with reference to the drawings, which are intended to illustrate, but not to limit, the disclosure. In the drawings, like reference characters denote corresponding features consistently throughout similar embodiments.

FIG. 1 is a perspective view of a dilation device according to an embodiment of the present disclosure.

FIG. 2 is a cross sectional view of the dilation device shown in FIG. 1.

FIG. 3 is a perspective view of the dilation device shown in FIG. 1 with implants positioned around an inflatable body.

FIG. 4 is a proximal end view of the dilation device shown in FIG. 1.

FIG. 5 is a distal end view of the dilation device shown in FIG. 1.

FIG. 6 is a cross sectional view of a dilation device according to an embodiment of the present disclosure.

FIG. 7 is a cross sectional view of a dilation device according to an embodiment of the present disclosure.

FIG. 8 is a cross sectional view of an implant according to an embodiment of the present disclosure.

FIG. 9 is a cross sectional view of a dilation device according to an embodiment of the present disclosure.

FIG. 10 is a side view of a deployment apparatus according to an embodiment of the present disclosure.

FIG. 11 is a side view of a deployment apparatus according to an embodiment of the present disclosure.

FIG. 12 is a side view of the deployment apparatus shown in FIG. 11 with a sheath retracted.

FIG. 13 is a side view of the deployment apparatus shown in FIG. 11 with a sheath retracted.

FIG. 14 is a schematic view of a deployment apparatus approaching an aortic valve.

FIG. 15 is a schematic view of a dilation device expanding aortic valve leaflets.

FIG. 16 is a schematic view of an implant in position within an aortic valve annulus.

FIG. 17 is a schematic view of an implant in position within an aortic valve annulus and a dilation device according to an embodiment of the present disclosure.

FIG. 18 is a schematic view of an implant expanded within an aortic valve annulus with a dilation device apparatus according to an embodiment of the present disclosure.

FIG. 19 is a schematic view of a deployed implant according to an embodiment of the present disclosure.

### DETAILED DESCRIPTION

The following description and examples illustrate some example embodiments of the disclosure in detail. Those of skill in the art will recognize that there are numerous variations and modifications of the disclosure that are encompassed by its scope. Accordingly, the description of a certain example embodiment should not be deemed to limit the scope of the present disclosure.

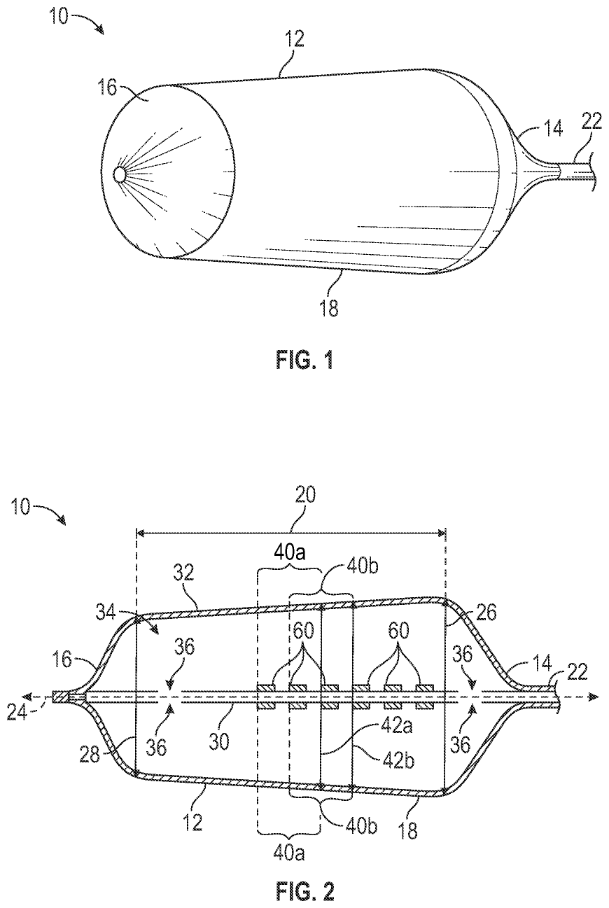

FIG. 1 illustrates a perspective view of a dilation device 10 for an implant, according to an embodiment of the present disclosure. The dilation device 10 may include an inflatable body 12 having a first end or proximal end 14, a second end or distal end 16, and a central body 18 positioned between the first or proximal end 14 and the second or distal end 16 and having a length 20 (marked in FIG. 2).

The first or proximal end 14 of the inflatable body 12 may be coupled to a shaft 22 of a deployment apparatus that may be utilized to deploy the inflatable body 12 and may be utilized to deploy an implant as well in various embodiments. The second or distal end 16 of the inflatable body 12 forms the opposite end of the inflatable body 12 and may include a leading tip of the dilation device 10.

The central body 18 may include a pressing portion of the inflatable body 12 that is configured to press an inner surface of an implant to dilate the implant. The central body 18 may press towards the implant to dilate the implant and apply a force to the implant to dilate the implant. The central body 18 may be configured with an outer surface configured for contact with the implant to press the implant. In various embodiments, the central body 18 may be covered with another structure and yet may press the inner surface of the implant through the other structure.

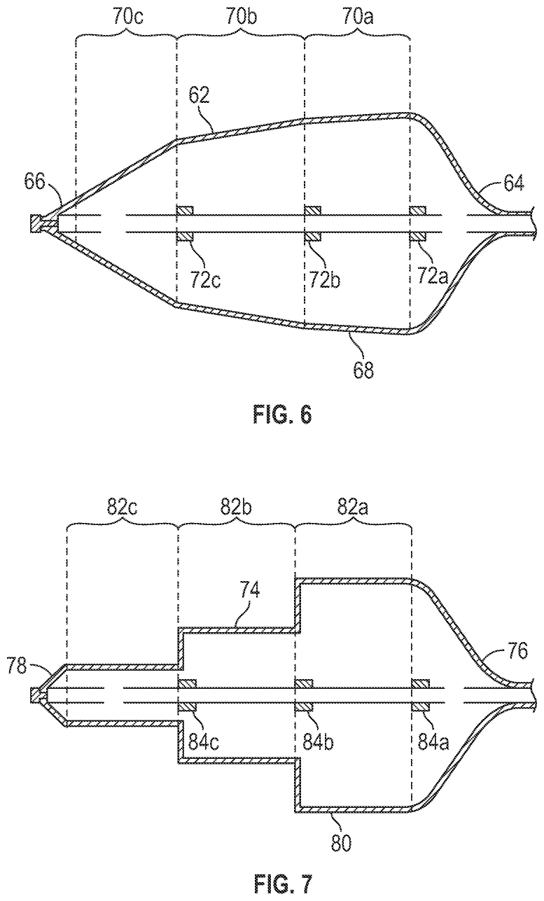

FIG. 2 illustrates a cross-sectional view of the dilation device 10 along a mid-line of the dilation device 10. The central body 18 is shown to have a profile that decreases in diameter along the length 20 of the central body 18 from the first or proximal end 14 to the second or distal end 16. The profile is a tapered profile. The profile is an angled profile that is angled downward in a direction towards the second or distal end 16. The profile angle may be a single angle that does not vary along the length of the central body 18. In various embodiments, for example as shown in FIG. 6, the angle may vary along the length of the central body 18. The profile of the central body 18 may have a gradual taper, such that the diameter 26 of the central body 18 proximate the first or proximal end 14 gradually decreases to the diameter 28 of the central body 18 proximate the second or distal end 16. The profile may be symmetrical about a longitudinal axis 24 that the central body 18 extends around.

The central body 18 may be shaped as a frustum as shown in FIGS. 1 and 2, which may include a conical frustum or may include another form of frustum in various embodiments (e.g., pyramidal or another shape).

The ends 14, 16 of the inflatable body 12 may have shapes that contour down to a diameter of a shaft or lumen that couples to the inflatable body 12. For example, the second or distal end 16 may couple to a distal portion of the central body 18 and may taper down to a smaller diameter than the diameter 28 of the distal portion of the central body 18 (for example, a diameter of the inflation lumen 30). The distal end 16 may be configured to be atraumatic if the distal end 16 were to contact a portion of a subject. The proximal end 14 may couple to a proximal portion of the central body 18 and may taper down to a smaller diameter than the diameter 26 of the proximal portion of the central body 18 (for example, a diameter of the shaft 22). The proximal end 14 may taper downward in a direction that is proximal from the proximal portion of the central body 18.

The inflatable body 12 may be formed by a wall 32 that may form the outer surface of the inflatable body 12 and may surround an interior cavity 34 that is configured to be filled with a fluid to inflate the inflatable body 12. The pressure of the fluid within the inflatable body 12 may provide the force that allows the central body 18 or pressing portion of the inflatable body 12 to dilate the implant. The interior cavity 34 is shown in FIG. 2 to comprise a single cavity, although in various embodiments other configurations may be utilized. For example, multiple chambers may be utilized which may be separate or may be in fluid connection with each other. In various embodiments, the inflatable body 12 may include separate bodies, such as separate inflatable bodies that may be inflated separately or may be inflated together.

The wall 32 of the inflatable body 12 may be configured such that the inflatable body 12 is non-compliant. As such, the inflatable body 12 may be configured to not expand upon meeting or exceeding an inflation pressure, and may be configured such that a defined profile of the inflatable body 12 does not change upon reaching or exceeding the inflation pressure. The inflatable body 12 may be preformed with a defined profile. The inflatable body 12 may be configured to not expand beyond the defined profile of the inflatable body 12 upon inflation of the inflatable body. For example, as the inflatable body 12 reaches the inflation pressure, the diameters 26, 28 of the inflatable body 12 may be configured to increase no further upon additional inflation pressure being provided. In various embodiments, the inflatable body 12 may be semi-compliant. In various embodiments, the inflatable body 12 may be compliant and the amount of fluid inflating the inflatable body 12 may be controlled to control a size of the inflatable body 12.

An inflation lumen 30 may be provided that is configured to inflate the inflatable body 12. The inflation lumen 30 may be configured to inflate the interior cavity 34 of the inflatable body 12. The inflation lumen 30 may extend within the interior cavity 34 of the inflatable body 12 and may include channels 36 configured for fluid to be passed into in order to fill the interior cavity 34 and thus inflate the inflatable body 12. Further, the inflation lumen 30 may be configured to withdraw fluid from the interior cavity 34 through the channels 36 to deflate the inflatable body 12. The inflation lumen 30 may be in fluid communication with a port 38 (marked in FIG. 11) that may be utilized to pass fluid into or out of the inflation lumen 30 to control the inflation of the inflatable body 12.

The inflatable body 12 may be configured to be in an undeployed configuration for being positioned in a lumen of a deployment apparatus and may be configured to be inflated to a deployed configuration as shown in FIGS. 1-5. The inflation lumen 30 may be utilized to inflate the inflatable body 12 to move the inflatable body 12 to the deployed configuration and may be utilized to deflate the inflatable body 12 to move the inflatable body 12 to the undeployed configuration.

The inflatable body 12 may include a plurality of segments along the length of the central body 18. The segments may include portions of the central body 18 that are each configured to press an inner surface of an implant to dilate the implant. The plurality of segments may each have a different profile than each other of the plurality of segments. The different profiles may be due to a different greatest diameter of each segment. For example, as shown in FIG. 2, each of the plurality of segments (e.g., segments 40a, 40b) has a greatest diameter that is different than a greatest diameter of each other of the plurality of segments. A segment 40a (indicated by the bracket) of the central body 18 has an angled profile with a greatest diameter 42a at a proximal portion of the segment 40a. A segment 40b (indicated by the bracket) of the central body 18 has an angled profile with a greatest diameter 42b at a proximal portion of the segment 40b that is greater than the greatest diameter 42a of the segment 40a. Such a feature in FIG. 2 is due to the continuous tapered profile of the central body 18 along the length 20 of the central body. An implant that surrounds segment 40b will thus be expanded to a greater diameter than an implant that surrounds segment 40a. In various embodiments, the plurality of segments may each have a different profile than each other of the plurality of segments, or, at least one of the plurality of segments may have a profile that is different than a profile of another of the plurality of segments. The segments of the central body 18 may overlap each other as indicated in FIG. 2 or may be separate from each other.

Each of the plurality of segments shown in FIG. 2 has a profile angle that is at a same angle as each other of the plurality of segments. Such a feature in FIG. 2 is due to the single profile angle of the central body 18 along the length 20 of the central body 18.

The segments may not only have differing profiles resulting in different greatest diameters, but may also have differing profile angles than each other in various embodiments, for example as shown in FIG. 6. In various embodiments, the segments may have different profiles by having a same greatest diameter yet having differing profile angles (e.g. extending outward to the same greatest diameter yet doing so at angles that differ). Further, in various embodiments the differing profiles may result from a differing shape of each segment, for example a segment may have a linear profile shape as shown in FIG. 2, yet another segment may have a curved shape or other shape in various embodiments.

The segments may be configured to expand an implant, or multiple different implants, to a variety of expanded diameters. For example, the inflatable body 12 may be configured to expand an implant to the greatest diameter 42a or to the greatest diameter 42b depending on the position of the implant upon the central body 18. If the implant is positioned upon segment 40a, then the implant will be expanded to the greatest diameter 42a. If the implant is positioned upon segment 40b, then the implant will be expanded to the greatest diameter 42b. In this manner, a user may select the extent to which an implant is expanded by selectively positioning the implant upon the central body 18.

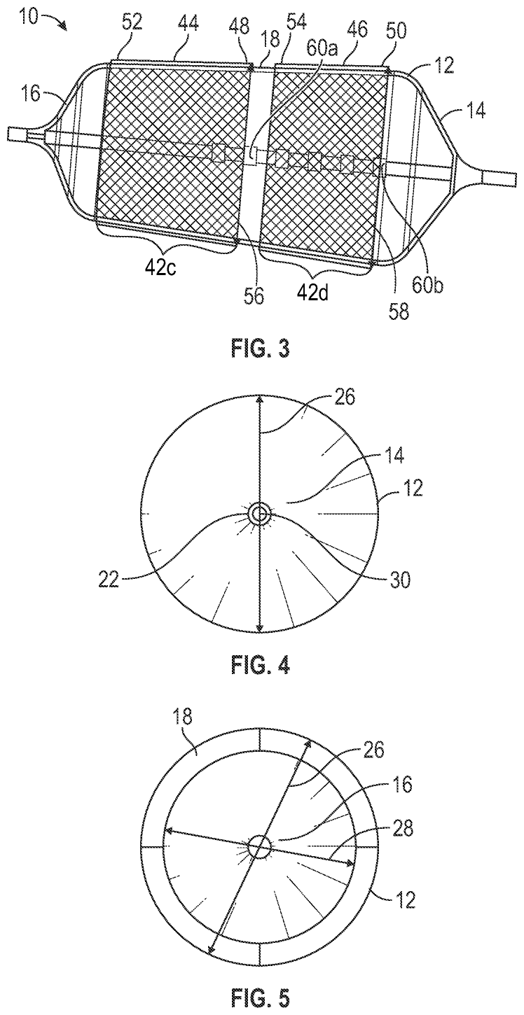

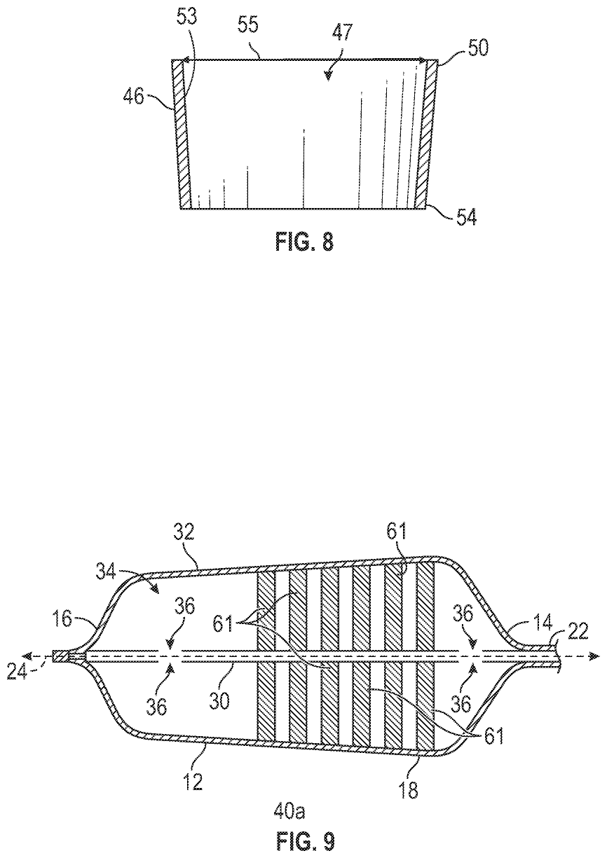

Further, the segments may be configured to have a profile that is angled to fit an angled interior profile of an implant. FIG. 8, for example, illustrates a cross sectional view of an embodiment of an implant 46, which is also shown in FIG. 3 positioned on the inflatable body 12. Features of the implant are not shown for clarity. For example, prosthetic heart valve leaflets may not be shown in FIG. 8 for clarity in an embodiment in which the implant 46 is a prosthetic heart valve. The implant 46 has an angled interior profile that faces an interior cavity 47 of the implant 46. The implant 46 has a second or distal end 54 that is narrower than a first or proximal end 50 and has the inner surface 53 forming an angled interior profile from the distal end 54 to the proximal end 50. The implant 46 may be considered a “V” shaped implant or otherwise an implant having a conical frustum interior shape or may include another form of frustum in various embodiments (e.g., pyramidal or another shape). The implant may have an outer profile that is the same as the interior profile, or may be different in various embodiments. The implant 46 may be configured to expand to an expanded interior diameter 55 as indicated in FIG. 8.

The segments of the central body 18 may have a profile that fits the interior profile of the implant 46. The profile angle of the respective segments may be at a same angle as a profile angle of the angled interior profile to fit the angled interior profile, or may be at a slightly different angle to still fit the angled interior profile. The fit to the angled interior profile may allow for a large contact surface area between the outer surface of the segment and the inner surface 53 of the implant. FIG. 3, for example, illustrates a perspective view of the inflatable body 12 including two implants 44, 46 extending around respective segments 42c, 42d of the central body 18. The central body 18 is positioned within the interior cavities of the implants 44, 46. The implants 44, 46 may each have a different diameter of expansion and may have a different interior profile of the implant 44, 46 due to the differing lengths of the implants 44, 46 and differing diameters at the respective proximal ends 48, 50 and distal ends 52, 54 of the implants 44, 46. The implant 44, for example, has a diameter at a proximal end 48 that is less then a diameter of a distal end 54 of the implant 46, which is less than the diameter of the proximal end 50 of the implant 46. The angle of the interior profile of the implants 44, 46 is the same.

The segments 42c, 42d may have a profile that is angled to fit an angled interior profile of the implants 44, 46. As such, as shown in FIG. 3, the profile of the segment 42c fits the angled interior profile of the implant 44 and the profile of the segment 42d fits the angled interior profile of the implant 46. A large contact surface area accordingly exists between the outer surface of the segment 42c, 42d and the inner surface of the respective implant 44, 46. The large contact surface area may improve the ability of the inflatable body 12 to apply a force to dilate the respective implant 44, 46.

As shown in FIG. 3, the inflatable body 12 may dilate implants to a variety of expanded diameters. The implant 44 may be dilated to a greatest diameter 56 of the distal segment 42c, and the implant 46 may be dilated to a greatest diameter 58 of the proximal segment 42d. As such, the same inflatable body 12 may be configured to dilate different implants to different expanded diameters. Further, the inflatable body 12 may be configured to dilate the same implant to different expanded diameters. The implant 44 for example, may be expanded to the diameter 56 and a user may determine a property of the implant 44 such as the quality level of deployment of the implant 44 at the diameter 56. If the user chooses to expand the implant 44 further, the inflatable body 12 may be deflated to reduce the force by the segment 42c upon the implant 44, and then the inflatable body 12 may be slid distally to position the implant 44 around segment 42d for example. The user may then inflate the inflatable body 12 again to expand the implant 44 to the diameter 58 if desired. As such, the inflatable body 12 may be utilized to progressively expand an implant by varying the position of the implant upon the central body 18.

The implants 44, 46 may each be configured to expand to an expanded interior diameter upon deployment. An expanded interior diameter 55 of implant 46 is shown in FIG. 8 for example. The central body 18 accordingly may include segments (as shown) that have a greatest diameter 56, 58 that is the same as the expanded interior diameter of the respective implants 44, 46. Further, with regard to implant 44, the central body 18 includes a segment 42d that has a greatest diameter that is greater than the expanded interior diameter of the implant 44. With regard to implant 46, the central body 18 includes a segment 42c that has a greatest diameter that is less than the expanded interior diameter of the implant 46. As such, a variety of implants having different expanded interior diameters may be dilated with the dilation device 10. Further the central body 18 has a length that is greater than the length of the implants 44, 46 to allow for variable positioning of the central body 18 relative to the implants 44, 46.

Referring back to FIG. 2, the dilation device 10 may include a plurality of imaging markers 60. The plurality of imaging markers 60 may each be configured to indicate a position of a respective one of the plurality of segments. The imaging markers 60 may include radiopaque markers or other forms of markers that are configured to be imaged. The imaging markers 60 for example, may include echogenic markers configured to be viewed on echocardiography or another form of radiography or ultrasound imaging. The imaging markers 60 may be configured to be viewed on fluoroscopy or another form of imaging. The imaging markers 60 may be spaced from each other along the length of the central body 18. The imaging markers 60 may be spaced from each other along the longitudinal axis 24 of the inflatable body 12. The imaging markers 60 may be equally spaced from each other or another spacing may be utilized. The imaging markers 60 may surround the longitudinal axis 24 of the inflatable body 12.

The imaging markers 60 may be positioned within the interior cavity 34 as shown in FIG. 2 and may be positioned on the inflation lumen 30. In various embodiments, the imaging markers 60 may be positioned in another location. For example, the imaging markers 60 may be positioned on the inflatable body 12 in various embodiments. FIG. 9, for example, illustrates an embodiment in which imaging markers 61 are positioned on the wall 32 of the inflatable body 12 and extend circumferentially around the axis 24. The imaging markers 61 for example may be inside the wall 32. The wall 32 may include multiple layers (e.g., double layers, triple layers, etc.) and imaging markers 61 may be positioned between such layers. The imaging markers 61 may also be positioned on the surface of the wall 32 (e.g., printed on the wall 32) if desired. Other configurations may be utilized as desired.

Referring to FIG. 3, the imaging markers 60 may be configured to indicate a position of a respective one of the plurality of segments by each imaging marker 60 corresponding to a position of the respective segment. A position of each of the plurality of imaging markers 60 may indicate a position of the respective one of the plurality of segments. An imaging marker 60a, for example, is positioned at a proximal portion of the segment 42c and thus may indicate the end of the segment 42c and accordingly the position of the segment 42c. Imaging marker 60b is positioned at a proximal portion of the segment 42d and thus may indicate the end of the segment 42d and accordingly the position of the segment 42d. Each of the plurality of imaging markers 60 may be configured to be located at a position relative to the implant to indicate the position of the respective one of the plurality of segments (e.g., such as at the segment or at an end portion of the segment, among other positions). A user accordingly during a deployment procedure may image the imaging markers 60a, b and align the implant 44 with the marker 60a so that the user knows that the implant 44 is positioned around segment 42c. The user may know the correspondence between the imaging markers 60 and the position of the segments, and may thus be able to position the implant 44 relative to the imaging markers 60 to assure that the implant 44 is upon the desired segment of the inflatable body 12.

As another example, upon the user aligning the implant 44 with the imaging marker 60a and dilating the implant 44 to the greatest diameter 56, the user may desire to dilate the implant further 44. The user may then deflate the inflatable body 12 and then slide the inflatable body 12 distally until the imaging marker 60b is aligned with the implant 44. The user may then dilate the implant 44 to the greatest diameter 58 (if the implant 44 for example were configured to expand to the greatest diameter 58). The user may count the number of imaging markers 60 between the marker 60a and marker 60b to recognize that the implant 44 extends around segment 42d. In various embodiments, the sequential number of the imaging marker 60 may indicate a greatest diameter or other property of the respective segment. For example, each imaging marker 60 sequentially in line may represent an additional millimeter of greatest diameter or another size of diameter. As such, a user may count three imaging markers to recognize that three additional or less millimeters of greatest diameter will be provided by that segment of the inflatable body 12. Additional properties of the inflatable body 12 may be indicated by the imaging markers 60. For example, an angle of the segments may be indicated by the imaging markers 60, with each imaging marker 60 sequentially in line representing an additional degree of angle of an angled profile or another variation in angle. The imaging markers 60 may directly indicate a property of the inflatable body 12, for example, by the imaging markers being formed in the shape of letters, words, numbers, or other markings indicating a property.

The imaging markers 60 may serve as discrete, spaced apart, graduation marks to align or realign the position of the inflatable body 12 relative to the implant.

FIG. 4 illustrates a first end view or proximal end view of the inflatable body 12. FIG. 5 illustrates a second end view or distal end view of the inflatable body 12.

FIG. 6 illustrates an embodiment of the inflatable body in which each of the plurality of segments has a profile angle that is at a different angle than a profile angle of each other of the plurality of segments. The inflatable body 62 is configured similarly as the inflatable body 12 and includes a first or proximal end 64, a second or distal end 66, and a central body 68 positioned between the first or proximal end 64 and the second or distal end 66 and having a length. The central body 68 may include a pressing portion of the inflatable body 62 that is configured to press an inner surface of an implant to dilate the implant. The central body 68 is shown to have a profile that decreases in diameter along the length of the central body 68 from the first or proximal end 64 to the second or distal end 66. The profile is a tapered profile and an angled profile, that is angled downward in a direction towards the distal end 66. The profile angle varies along the length of the central body.

At least one of the plurality of segments has a profile angle that is at a different angle than a profile angle of another of the plurality of segments. For example, a segment 70b may have a profile angle that is at a greater angle than a profile angle of adjacent segment 70a. Further, a segment 70c may have a profile angle that is at a greater angle than a profile angle of adjacent segment 70b and of segment 70a. Each segment 70a—c also has a greater diameter that is different than the greatest diameter of the other segments. The varied angles of the segments 70a—c may be utilized to dilate various implants having profile angles that fit the profile angles of the respective segment 70a—c. For example, the same inflatable body 62 may be used to dilate different implants. Further, in various embodiments, the varied angles of the segments 70a—c may be utilized to dilate the same implant, yet at a different angle if so desired by the user. For example, the user may desire to initially dilate an implant at a high angle segment 70c to rapidly dilate the implant. The user may then desire to dilate the same implant at a low angle segment 70a to more slowly dilate the implant at the later stages of deployment.

Imaging markers 72a—c may be utilized in a similar manner as imaging markers 60, to indicate the position of respective segments 70a—c.

FIG. 7 illustrates an embodiment of the inflatable body in which each segment has a different greatest diameter than the other segments. The inflatable body 74 is configured similarly as the inflatable body 12 and includes a first or proximal end 76, a second or distal end 78, and a central body 80 positioned between the first or proximal end 76 and the second or distal end 78 and having a length. The central body 80 may include a pressing portion of the inflatable body 74 that is configured to press an inner surface of an implant to dilate the implant. The central body 80 is shown to have a profile that decreases in diameter along the length of the central body 80 from the proximal end 76 to the distal end 78.

The profile is a stepped profile. For example, each segment 82a—c has a constant diameter along its length, and at least one of the plurality of segments 82a—c has a different diameter than another of the plurality of segments. Further, each of the segments 82a—c has a different diameter than each other of the segments 82a—c. The segment 82b has a profile with a greatest diameter that is less than the greatest diameter of the segment 82a. The segment 82c has a profile with a greatest diameter that is less than the greatest diameter of the segment 82b. The varied diameters of the segments 82a—c may be utilized to dilate various implants to differing diameters. Further, in various embodiments, the segments 82a—c may be utilized to dilate the same implant to various diameters. The implant may have an interior profile that has a constant diameter, and thus each segment 82a—c has a profile that fits the constant diameter profile of that implant. However, the segments 82a—c may also be utilized to dilate an implant having an angled profile if desired.

Imaging markers 84a—c may be utilized in a similar manner as imaging markers 60, to indicate the respective position of segments 82a—c.

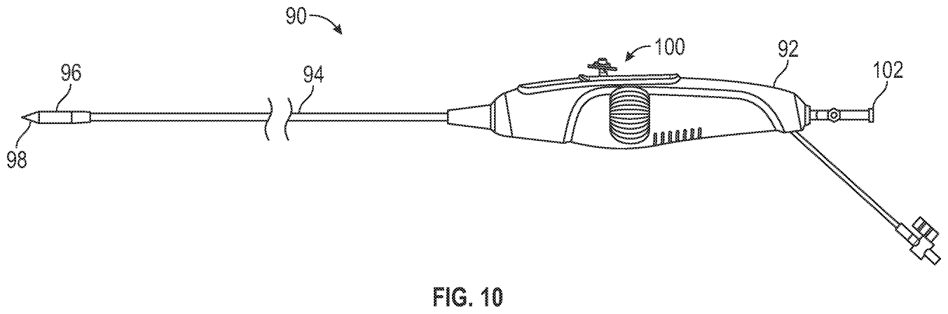

The inflatable bodies disclosed herein may be delivered to a desired deployment location of the implant via a deployment apparatus that may be dedicated for use with the inflatable bodies, or may be delivered via a deployment apparatus for the implant. FIG. 10, for example, illustrates an embodiment of a deployment apparatus 90 for an implant. The deployment apparatus 90 may include a handle 92, an elongate shaft 94 coupled to the handle 92 and an implant retention area surrounded by a capsule 96 at an end of the elongate shaft 94. The elongate shaft 94 may include a nose cone 98 at a distal end of the capsule 96. The deployment apparatus 90 may include an actuation mechanism 100 for actuating operation of the deployment apparatus 90, which may include releasing the implant from the capsule 96, and may include deflecting the elongate shaft 94 into a desired orientation. The deployment apparatus 90 may further include a dilation device for dilating a native heart valve or for initially dilating an implant.

The inflatable bodies may be coupled to the deployment apparatus 90, for example coupled to the elongate shaft 94. The inflation lumen 30 shown in FIG. 2 for example may extend through a portion of the deployment apparatus 90 and may be accessed via a port 102 or via another means.

FIG. 11 illustrates an embodiment of a deployment apparatus 104 that may be utilized without a feature for releasing the implant. The deployment apparatus 104 may include an elongate shaft 106 having a sheath 108 covering the implant. A proximal end of the deployment apparatus 104 may include the port 38 for accessing the inflation lumen. The elongate shaft 106 may be configured to be flexible to deflect to the desired portion of the subject. The elongate shaft 106 may be steerable such as with an actuation mechanism that may be configured similarly as the actuation mechanism 100 shown in FIG. 10.

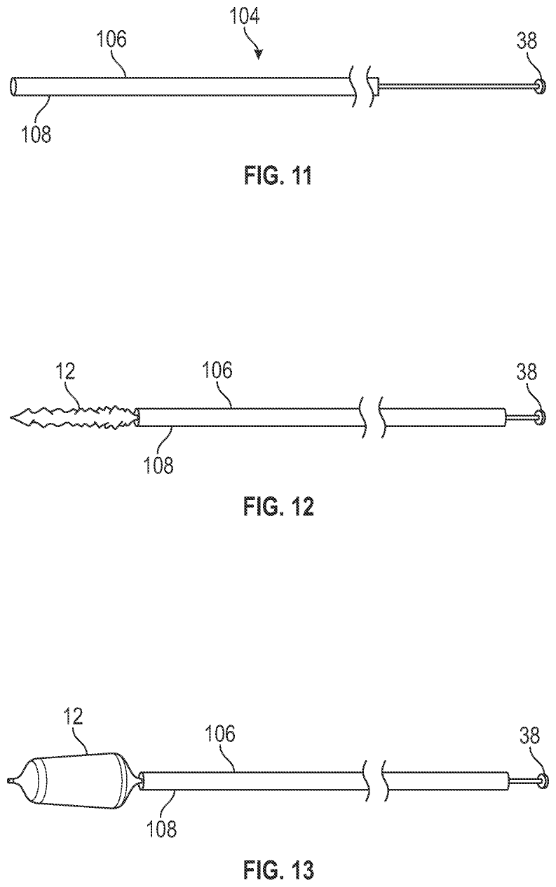

Referring to FIG. 12, the sheath 108 may be retracted to expose the inflatable body 12. The inflatable body 12 may be in a undeployed, deflated, or unexpanded state. As shown, in such a configuration the inflatable body 12 is configured to be positioned in a lumen of a deployment apparatus. The inflatable body 12 may be inflated via the port 38 to move the inflatable body 12 to the deployed, inflated, or expanded state having a larger diameter and size than in the undeployed, deflated, or unexpanded state as shown in FIG. 13 for example. The inflatable body 12 may then be deflated, and the sheath 108 may extend over the inflatable body 12 to cover the inflatable body 12.

The configuration of the deployment apparatus may vary from the configuration shown in FIGS. 10-13. Other types of deployment apparatuses may be utilized than shown in FIG. 10-13.

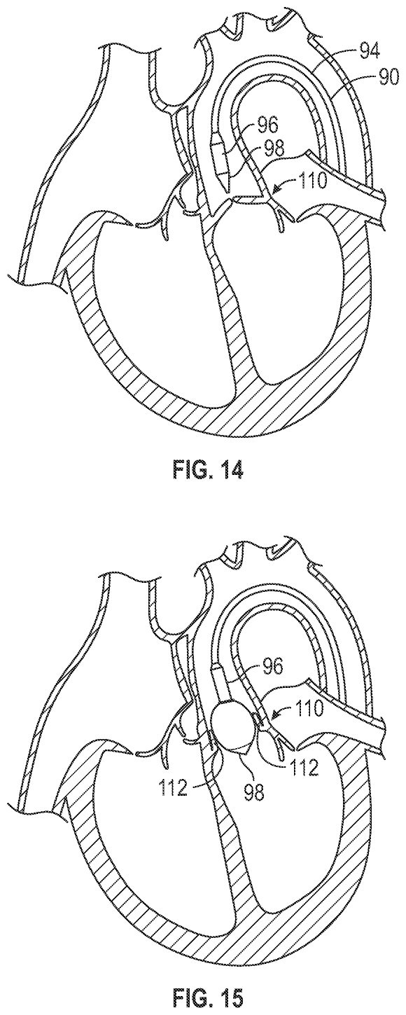

FIGS. 14-19 illustrate an exemplary method of utilizing the dilation devices disclosed herein. Methods disclosed herein may vary from the steps shown in FIGS. 14-19. Referring to FIG. 14, the dilation device may be utilized in the deployment of an implant that is a prosthetic heart valve. The prosthetic heart valve may include a prosthetic aortic heart valve, or in various embodiments may include another form of prosthetic heart valve such as a mitral, tricuspid, or pulmonary heart valve. The implant in various embodiments may be utilized for repair, which may include repair of a portion of a heart, including heart valve repair. The implant in various embodiments may include other forms of medical implants including stents, among others. The implant may be configured to have an interior cavity for the inflatable bodies disclosed herein to be positioned in. The method may include dilating an implant utilizing an inflatable body having a central body pressed towards an inner surface of the implant. In various embodiments, other methods may be utilized.

FIG. 14 illustrates a step in a method of replacing an aortic heart valve. A deployment apparatus 90 for example as shown in FIG. 10 may be utilized to approach the native aortic heart valve 110. The elongate shaft 94 may be deflected to allow the capsule 96 to approach the native aortic heart valve 110 through the aortic arch.

Upon the elongate shaft 94 reaching the desired position within the heart, the capsule 96 may be retracted. Referring to FIG. 15, a dilation device in the form of an inflatable body may be inflated to press against the native heart valve leaflets 112 to expand the aortic annulus. In various embodiments, the embodiments of dilation devices disclosed herein may be utilized to perform such an operation.

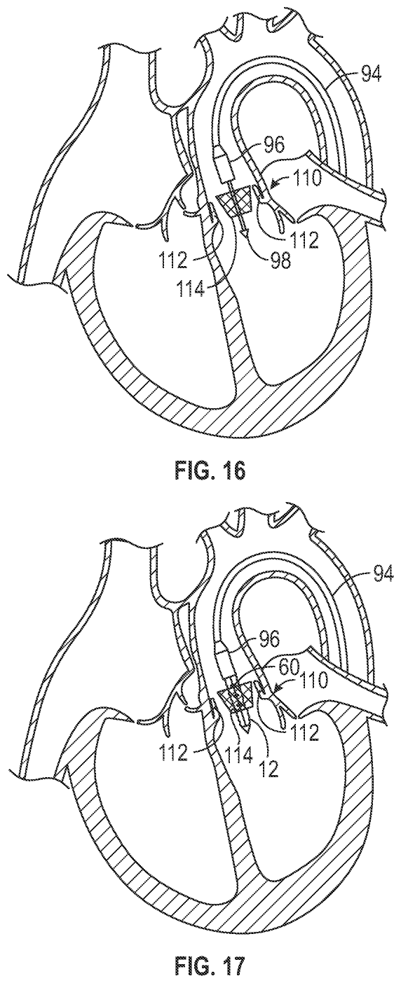

Referring to FIG. 16, an implant 114 may then be partially deployed, which may include releasing the implant 114 from the capsule 96. The implant 114 may not yet be in a fully expanded or deployed configuration, and a user may be able to position the implant 114 axially within the aortic annulus. The implant 114 as shown, comprises a replacement aortic heart valve. The implant 114 has an angled profile, similar to the profile of the implants 44, 46 shown in FIG. 3. The implant 114 may be configured similarly as the implants 44, 46 shown in FIG. 3.

Referring to FIG. 16, an inflatable body disclosed herein, such as inflatable body 12 may be positioned within the interior cavity of the implant 114. The inflatable body 12 may be in an undeployed, unexpanded, or uninflated state and thus the position of the inflatable body 12 may be configured to vary axially relative to the implant 114. The user may be able to select one of the segments of the inflatable body 12 for the implant 114 to be dilated with. The user may select such a segment based on a variety of criteria disclosed herein, which may include a desired expansion diameter of the implant 114 or a profile of the implant 114. For example, in an embodiment in which the implant 114 is to expand to a certain diameter, a segment of the inflatable body 12 having the corresponding diameter is used to dilate the implant 114. A user may select the segment for dilating the implant based on a desired expanded diameter of the implant. Further, in an embodiment in which the implant 114 has a certain interior angled profile, a segment of an inflatable body fitting such a profile may be used to dilate the implant 114.

The plurality of imaging markers 60 may be imaged to determine which segment of the inflatable body 12 will dilate the implant 114. For example, fluoroscopy or echocardiography or another imaging method may be utilized. A user may identify at least one of the plurality of segments based on a plurality of imaging markers 60 that have been imaged. The user may examine the imaging markers 60 and select which segment of the inflatable body 12 will dilate the implant 114 by sliding the uninflated inflatable body 12 relative to the implant 114 to a defined location relative to the implant 114. The implant may be aligned with at least one of the plurality of imaging markers 60. For example, as shown in FIG. 17, an imaging marker 60 may be aligned with a proximal portion of the implant 114 corresponding to a segment to be dilated to expand the implant 114. The uninflated inflatable body 12 may be slide until the desired imaging marker 60 is aligned with the portion of the implant 114 and the segment of the inflatable body 12 is selected.

A segment of the inflatable body 12 may be used to dilate the implant 114 based on the local anatomy (for example aortic annulus size) proximate the deployment site.

In various embodiments, a segment of the uninflated inflatable body 12 for may be used an initial dilation operation, and then a second segment of the uninflated inflatable body 12 may be used for a second dilation operation, and so forth. As such, the implant 114 may be progressively dilated with various segments of the inflatable body 12. In various embodiments, a user may progressively inflate the inflatable body 12 and check the diameter and desired position of the implant 114, and may then continue to inflate the inflatable body 12 in a series of steps.

The inflatable body 12 may be utilized in a procedure of balloon post-dilation in which the implant 114 may be previously expanded via a separate dilation device.

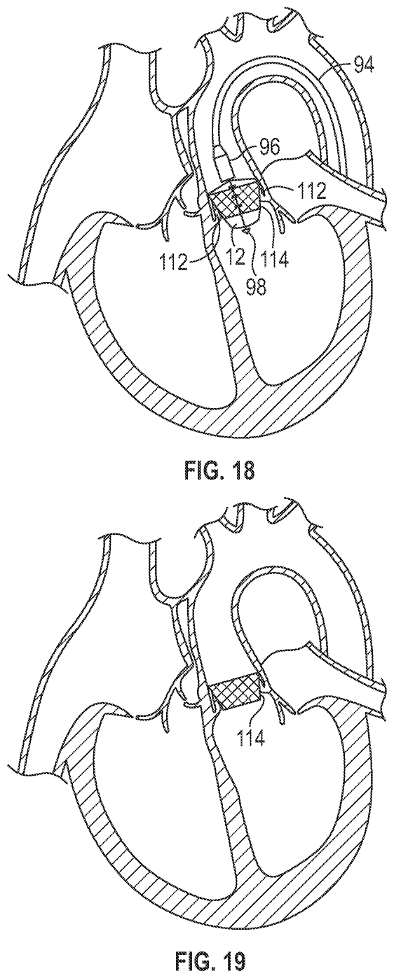

The inflatable body 12 may ultimately be inflated as shown in FIG. 18. The inflation may include inflating the selected one of the plurality of segments to the greatest diameter of the selected one of the plurality of segments to dilate the implant. As discussed, the inflatable body 12 may be non-compliant, such that the inflatable body 12 will not expand further than a defined diameter of the inflatable body 12. The selected one of the plurality of segments may be configured to not inflate beyond the greatest diameter of the selected one of the plurality of segments. Accordingly, the inflatable body 12 may be fully inflated via a port (for example port 102 as shown in FIG. 10) and the inflatable body 12 may have a precise diameter to expand the implant 114. Thus, there may be reduced cause for concern regarding overexpanding the implant 114 or regarding underexpanding the implant 114 based on an imprecisely inflated inflatable body 12. Further, in an embodiment in which the central body 18 of the inflatable body 12 has a profile that is angled to fit the angled interior profile of the implant 114, then a relatively large contact surface area may be utilized to uniformly apply a dilation force to the implant 114.

The inflatable body 12 may then be deflated and may be withdrawn. The inflatable body 12 may be withdrawn along with the deployment apparatus 90. In an embodiment in which a separate deployment apparatus is utilized for the inflatable body 12, then such a deployment apparatus may be withdrawn as well.

Referring to FIG. 19, the implant 114 may remain deployed within the aortic valve annulus. Other forms of methods may be utilized as desired.

The steps disclosed herein are disclosed with regard to the inflatable body 12, however, other forms of dilatation devices and inflatable bodies disclosed herein may be utilized in a similar manner. The deployment apparatus shown in FIG. 10 is disclosed as being utilized, however, other forms of deployment apparatuses may be utilized, for example, the deployment apparatus shown in FIGS. 11-13, among others. A separate deployment apparatus may be utilized to deploy the dilation devices disclosed herein than is utilized to deliver the implant to the desired location within the subject. The features of the deployment apparatus shown in FIGS. 11-13, such as the sheath, inflation lumen, port, and dilation device retention area may be utilized with any embodiment of deployment apparatus for the systems and methods disclosed herein.

The steps disclosed herein are disclosed with regard to deployment of a prosthetic aortic valve, however the steps may be utilized for a variety of other forms of heart valves and for other forms of implants. The implants may include stents or filters, or diagnostic devices, among others. The deployment apparatuses disclosed herein may be utilized to deploy the implant as well as utilize and deploy the embodiments of dilation devices disclosed herein. The implant, for example, may extend around the shaft 22 shown in FIG. 2 and may be deployed from a deployment apparatus that includes the dilation devices disclosed herein. The dilation devices disclosed herein may be coupled to a deployment apparatus such as an apparatus shown in FIG. 10, which may deploy the dilation devices.

The deployment apparatuses may be configured to deploy implants in the form of prosthetic heart valves, or may be configured to deploy the other forms of implants such as stents or filters, or diagnostic devices, among others.

The other forms of implants such as stents or filters, among others, may be configured similarly as the implants disclosed herein. For example, the implants utilized according to embodiments herein may have an angled interior profile as discussed herein, or may have other profiles as desired. The implants may have a uniform interior profile in various embodiments, for example. The implants may be configured to expand radially outward from an axis that the implant surrounds, for example a longitudinal axis of the implant.

In various embodiments, the orientation of the inflatable bodies disclosed herein may be reversed relative to a deployment apparatus. Thus, in an embodiment as shown in FIG. 1 for example, the inflatable body 12 may be reversed such that the first end or proximal end 14 is configured to face distally and the second end or distal end 16 is configured to face proximally. As such, in such a configuration, the first end or proximal end 14 is a distally facing end, and the second end or distal end 16 is a proximally facing end. Any embodiment of inflatable body disclosed herein may be utilized in such a manner. In such a configuration, an opposite orientation of an implant may be provided. As such, other forms of delivery may be utilized, such as transapical deployment (such as to an aortic valve), or deployment of an implant to a native valve having an opposite flow through the native valve, such as transseptal delivery to a native mitral valve. Thus, in various embodiments, the orientation of the inflatable bodies and implant may be reversed from the positions shown herein as desired to produce a desired result.

The systems disclosed herein may include implant deployment systems or other forms of systems that may utilize the components disclosed herein. For example, an implant and an inflatable body as disclosed herein may include an implant deployment system. An inflatable body as disclosed herein and a deployment apparatus may include an implant deployment system. Other forms of systems may be provided. Components disclosed herein may be utilized separately or in combination in various embodiments.

An inflatable body may be utilized in a variety of subjects and procedures. Subjects include (but are not limited to) medical patients, veterinary patients, animal models, cadavers, and simulators of the cardiac and vasculature system (e.g., anthropomorphic phantoms and explant tissue). Procedures include (but are not limited to) medical and training procedures.

The deployment apparatus and apparatuses and the systems disclosed herein may be used in a variety of procedures, which may include transcatheter aortic valve implantation (TAVI). The deployment apparatus and the systems disclosed herein may be utilized for transarterial access, including transfemoral access, to a subject's heart. In various embodiments, the deployment apparatus may be utilized for mitral, tricuspid, and pulmonary replacement and repair as well. The deployment systems may be utilized in transcatheter percutaneous procedures, including transarterial procedures, which may be transfemoral or transjugular. Transapical procedures, among others, may also be utilized.

Methods as disclosed herein may be utilized in locations that do not utilize native valves, including a pulmonary artery and in the vena cava, among other locations (other arteries, blood vessels, or other vasculature, among other portions of a subject). An implant such as a stent or other form of implant may be delivered to such portions of the subject.

The user as disclosed herein may include a surgeon, physician, or other medical professional, among other users.

Features of embodiments may be modified, substituted, excluded, or combined.

In addition, the methods herein are not limited to the methods specifically described, and may include methods of utilizing the systems and apparatuses disclosed herein.

The steps of the method may be modified, excluded, or added to, with systems, apparatuses, and methods disclosed herein.

The features of the embodiments disclosed herein may be implemented independently of the deployment apparatuses, or independent of other components disclosed herein. The various apparatuses of the systems may be implemented independently.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of systems, apparatuses, and methods as disclosed herein, which is defined solely by the claims. Accordingly, the systems, apparatuses, and methods are not limited to that precisely as shown and described.

Certain various embodiments of systems, apparatuses, and methods are described herein, including the best mode known to the inventors for carrying out the same. Of course, variations on these described embodiments will become apparent to those of ordinary skill in the art upon reading the foregoing description. The inventor expects skilled artisans to employ such variations as appropriate, and the inventors intend for the systems, apparatuses, and methods to be practiced otherwise than specifically described herein. Accordingly, the systems, apparatuses, and methods include all modifications and equivalents of the subject matter recited in the claims appended hereto as permitted by applicable law. Moreover, any combination of the above-described embodiments in all possible variations thereof is encompassed by the systems, apparatuses, and methods unless otherwise indicated herein or otherwise clearly contradicted by context.

Groupings of alternative embodiments, elements, or steps of the systems, apparatuses, and methods are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses an approximation that may vary, yet is capable of performing the desired operation or process discussed herein.

The terms “a,” “an,” “the” and similar referents used in the context of describing the systems, apparatuses, and methods (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the systems, apparatuses, and methods and does not pose a limitation on the scope of the systems, apparatuses, and methods otherwise claimed. No language in the present specification should be construed as indicating any non-claimed element essential to the practice of the systems, apparatuses, and methods.

All patents, patent publications, and other publications referenced and identified in the present specification are individually and expressly incorporated herein by reference in their entirety for the purpose of describing and disclosing, for example, the compositions and methodologies described in such publications that might be used in connection with the systems, apparatuses, and methods. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### EXEMPLARY EMBODIMENTS

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A dilation device for an implant, the dilation device comprising:

an inflatable body having a first end and a second end and a central body positioned between the first end and the second end and having a length, the central body configured to press an inner surface of the implant to dilate the implant and having a profile that decreases in diameter along the length of the central body from the first end to the second end.

Example 2. The dilation device of example 1, wherein the profile is a tapered profile.

Example 3. The dilation device of example 1 or example 2, wherein the profile is an angled profile.

Example 4. The dilation device of any of examples 1-3, wherein the central body includes a plurality of segments, at least one of the plurality of segments having a profile angle that is at a different angle than a profile angle of another of the plurality of segments.

Example 5. The dilation device of any of examples 1-4, wherein the central body includes a plurality of segments, each of the plurality of segments having a profile angle that is at a same angle as each other of the plurality of segments.

Example 6. The dilation device of example 4 or example 5, wherein each of the plurality of segments has a greatest diameter that is different than a greatest diameter of each other of the plurality of segments.

Example 7. The dilation device of any of examples 1-6, wherein the central body includes a plurality of segments; and further comprising a plurality of imaging markers each configured to indicate a position of a respective one of the plurality of segments.

Example 8. The dilation device of example 7, wherein at least one of the plurality of imaging markers comprises radiopaque markers.

Example 9. The dilation device of example 7 or example 8, wherein at least one of the plurality of imaging markers is positioned within an interior cavity of the inflatable body or is positioned on a wall of the inflatable body.

Example 10. The dilation device of any of examples 1-9, wherein the central body includes a plurality of segments each having a constant diameter, at least one of the plurality of segments having a different diameter than another of the plurality of segments.

Example 11. The dilation device of any of examples 1-10, wherein the inflatable body is non-compliant.

Example 12. The dilation device of any of examples 1-10, wherein the inflatable body is semi-compliant.

Example 13. The dilation device of any of examples 1-12, wherein the inflatable body is coupled to an elongate shaft of a deployment apparatus for deploying the implant, and the inflatable body is configured to be in an undeployed configuration and is configured to be inflated to a deployed configuration, and further comprising an inflation lumen for inflating an interior cavity of the inflatable body.

Example 14. The dilation device of any of examples 1-13, wherein the inflatable body is configured to not expand beyond a defined profile of the inflatable body upon inflation of the inflatable body.

Example 15. The dilation device of any of examples 1-14, wherein the first end of the inflatable body is a proximal end and the second end of the inflatable body is a distal end.

Example 16. A dilation device for an implant, the dilation device comprising:

an inflatable body having a first end and a second end and a central body positioned between the first end and the second end and configured to press an inner surface of the implant to dilate the implant, the central body including a plurality of segments each having a different profile than each other of the plurality of segments; and a plurality of imaging markers, wherein each imaging marker is configured to be indicative of a position of a segment of the plurality of segments.

Example 17. The dilation device of example 16, wherein the plurality of imaging markers comprises radiopaque markers.

Example 18. The dilation device of example 16 or example 17, wherein the imaging markers are spaced from each other along a longitudinal axis of the inflatable body.

Example 19. The dilation device of any of examples 16-18, wherein at least one of the plurality of segments has a profile angle that is at a different angle than a profile angle of another of the plurality of segments.

Example 20. The dilation device of any of examples 16-18, wherein each of the plurality of segments has a profile angle that is at a same angle as each other of the plurality of segments.

Example 21. The dilation device of any of examples 16-20, wherein each of the plurality of segments has a greatest diameter that is different than a greatest diameter of each other of the plurality of segments.

Example 22. The dilation device of any of examples 16-21, wherein a position of each of the plurality of imaging markers indicates the position of the respective one of the plurality of segments.

Example 23. The dilation device of any of examples 16-22, wherein the central body has a tapered profile.

Example 24. The dilation device of any of examples 16-23, wherein the central body has an angled profile.

Example 25. The dilation device of any of examples 16-24, wherein the plurality of imaging markers are positioned within an interior cavity of the inflatable body or are positioned on a wall of the inflatable body.

Example 26. The dilation device of any of examples 16-25, wherein the inflatable body is non-compliant.

Example 27. The dilation device of any of examples 16-25, wherein the inflatable body is semi-compliant.

Example 28. The dilation device of any of examples 16-27, wherein the inflatable body is coupled to an elongate shaft of a deployment apparatus for deploying the implant, and the inflatable body is configured to be in an undeployed configuration and is configured to be inflated to a deployed configuration, and further comprising an inflation lumen for inflating an interior cavity of the inflatable body.

Example 29. The dilation device of any of examples 16-28, wherein the inflatable body is configured to not expand beyond a defined profile of the inflatable body upon inflation of the inflatable body.

Example 30. The dilation device of any of examples 16-29, wherein the first end of the inflatable body is a proximal end and the second end of the inflatable body is a distal end.

Example 31. An implant deployment system comprising:
an implant configured to be deployed to a deployment site within a subject and having an angled interior profile that faces an interior cavity of the implant; and
an inflatable body having a central body configured to be positioned within the interior cavity of the implant and configured to press towards the implant to dilate the implant, the central body having a profile that is angled to fit the angled interior profile of the implant.

Example 32. The implant deployment system of example 31, wherein the implant has a length and the central body has a length that is greater than the length of the implant.

Example 33. The implant deployment system of example 31 or example 32, wherein the central body includes a plurality of segments each having a different profile than each other of the plurality of segments.

Example 34. The implant deployment system of example 33, wherein the implant is configured to expand to an expanded interior diameter, and at least one of the plurality of segments has a greatest diameter that is the same as the expanded interior diameter.

Example 35. The implant deployment system of example 33 or example 34, wherein the implant is configured to expand to an expanded interior diameter, and at least one of the plurality of segments has a greatest diameter that is greater than the expanded interior diameter.

Example 36. The implant deployment system of any of examples 33-35, wherein the implant is configured to expand to an expanded interior diameter, and at least one of the plurality of segments has a greatest diameter that is less than the expanded interior diameter.

Example 37. The implant deployment system of any of examples 33-36, further comprising a plurality of imaging markers each configured to indicate a position of a respective one of the plurality of segments.

Example 38. The implant deployment system of example 37, wherein each of the plurality of imaging markers are configured to be located at a position relative to the implant to indicate the position of the respective one of the plurality of segments.

Example 39. The implant deployment system of any of examples 31-38, wherein the profile of the central body has a profile angle that is at a same angle as a profile angle of the angled interior profile.

Example 40. The implant deployment system of any of examples 31-39, wherein the implant comprises a prosthetic heart valve.

Example 41. The implant deployment system of any of examples 31-40, further comprising a deployment apparatus having an elongate shaft coupled to the inflatable body.

Example 42. The implant deployment system of any of examples 31-41, wherein the inflatable body is non-compliant.

Example 43. The implant deployment system of any of examples 31-41, wherein the inflatable body is semi-compliant.

Example 44. The implant deployment system of any of examples 31-43, wherein the inflatable body is configured to be in an undeployed configuration and is configured to be inflated to a deployed configuration.

Example 45. The implant deployment system of any of examples 31-44, wherein the inflatable body is configured to not expand beyond a defined profile of the inflatable body upon inflation of the inflatable body.

Example 46. An implant deployment system comprising:
a deployment apparatus for deploying an implant and having an elongate shaft; and
an inflatable body coupled to the elongate shaft and having a first end and a second end and a central body positioned between the first end and the second end and having a length, the central body configured to press an inner surface of the implant to dilate the implant and having a profile that decreases in diameter along the length of the central body from the first end to the second end.

Example 47. The implant deployment system of example 46, wherein the profile is a tapered profile.

Example 48. The implant deployment system of example 46 or example 47, wherein the profile is an angled profile.

Example 49. The implant deployment system of any of examples 46-48, wherein the central body includes a plurality of segments, at least one of the plurality of segments having a profile angle that is at a different angle than a profile angle of another of the plurality of segments.

Example 50. The implant deployment system of any of examples 46-49, wherein the central body includes a plurality of segments, each of the plurality of segments having a profile angle that is at a same angle as each other of the plurality of segments.

Example 51. The implant deployment system of example 49 or example 50, wherein each of the plurality of segments has a greatest diameter that is different than a greatest diameter of each other of the plurality of segments.

Example 52. The implant deployment system of any of examples 46-51, wherein the central body includes a plurality of segments, and further comprising a plurality of imaging markers each configured to indicate a position of a respective one of the plurality of segments.

Example 53. The implant deployment system of example 52, wherein the plurality of imaging markers comprise radiopaque markers.

Example 54. The implant deployment system of example 52 or example 53, wherein the plurality of imaging markers are positioned within an interior cavity of the inflatable body or are positioned on a wall of the inflatable body.

Example 55. The implant deployment system of any of examples 46-54, wherein the central body includes a plurality of segments each having a constant diameter, at least one of the plurality of segments having a different diameter than another of the plurality of segments.

Example 56. The implant deployment system of any of examples 46-55, wherein the inflatable body is non-compliant.

Example 57. The implant deployment system of any of examples 46-55, wherein the inflatable body is semi-compliant.

Example 58. The implant deployment system of any of examples 46-57, wherein the inflatable body is configured to be in an undeployed configuration and is configured to be inflated to a deployed configuration, and further comprising an inflation lumen for inflating an interior cavity of the inflatable body.

Example 59. The implant deployment system of any of examples 46-58, further comprising the implant, and wherein the central body has a profile that is angled to fit an angled interior profile of the implant.

Example 60. The implant deployment system of any of examples 46-59, wherein the first end of the inflatable body is a proximal end and the second end of the inflatable body is a distal end.

Example 61. An implant deployment system comprising:
a deployment apparatus for deploying an implant and having an elongate shaft;
an inflatable body coupled to the elongate shaft and having a first end and a second end and a central body positioned between the first end and the second end and configured to press an inner surface of the implant to dilate the implant, the central body including a plurality of segments each having a different profile than each other of the plurality of segments; and
a plurality of imaging markers coupled to the elongate shaft and each configured to indicate a position of a respective one of the plurality of segments.

Example 62. The implant deployment system of example 61, wherein the plurality of imaging markers comprise radiopaque markers.

Example 63. The implant deployment system of example 61 or example 62, wherein the plurality of imaging markers are spaced from each other along a longitudinal axis of the inflatable body.

Example 64. The implant deployment system of any of examples 61-63, wherein at least one of the plurality of segments has a profile angle that is at a different angle than a profile angle of another of the plurality of segments.

Example 65. The implant deployment system of any of examples 61-63, wherein each of the plurality of segments has a profile angle that is at a same angle as each other of the plurality of segments.

Example 66. The implant deployment system of any of examples 61-65, wherein each of the plurality of segments has a greatest diameter that is different than a greatest diameter of each other of the plurality of segments.

Example 67. The implant deployment system of any of examples 61-66, wherein a position of each of the plurality of imaging markers indicates the position of the respective one of the plurality of segments.

Example 68. The implant deployment system of any of examples 61-67, wherein the central body has a tapered profile.

Example 69. The implant deployment system of any of examples 61-68, wherein the central body has an angled profile.

Example 70. The implant deployment system of any of examples 61-69, wherein the plurality of imaging markers are positioned within an interior cavity of the inflatable body or are positioned on a wall of the inflatable body.

Example 71. The implant deployment system of any of examples 61-70, wherein the inflatable body is non-compliant.

Example 72. The implant deployment system of any of examples 61-70, wherein the inflatable body is semi-compliant.

Example 73. The implant deployment system of any of examples 61-72, wherein the inflatable body is configured to be in an undeployed configuration and is configured to be inflated to a deployed configuration, and further comprising an inflation lumen for inflating an interior cavity of the inflatable body.

Example 74. The implant deployment system of any of examples 61-73, further comprising the implant, and wherein the central body has a profile that is angled to fit an angled interior profile of the implant.

Example 75. The implant deployment system of any of examples 61-74, wherein the first end of the inflatable body is a proximal end and the second end of the inflatable body is a distal end.

Example 76. A method comprising:
dilating an implant utilizing an inflatable body having a central body pressed towards an inner surface of the implant, the inflatable body having a first end and a second end and a central body positioned between the first end and the second end, the central body having a length and having a profile that decreases in diameter along the length of the central body from the first end to the second end.

Example 77. The method of example 76, wherein the central body includes a plurality of segments, and the method further comprises selecting one of the plurality of segments for dilating the implant.

Example 78. The method of example 77, wherein each of the plurality of segments has a greatest diameter that is different than a greatest diameter of each other of the plurality of segments.

Example 79. The method of example 77 or example 78, further comprising selecting one of the plurality of segments for dilating the implant based on a diameter to dilate the implant to.

Example 80. The method of any of examples 77-79, further comprising inflating the selected one of the plurality of segments to a greatest diameter of the selected one of the plurality of segments to dilate the implant.

Example 81. The method of any of examples 77-80, wherein the selected one of the plurality of segments is configured to not inflate beyond a greatest diameter of the selected one of the plurality of segments.

Example 82. The method of any of examples 77-81, further comprising identifying at least one of the plurality of segments based on a plurality of imaging markers that have been imaged.

Example 83. The method of example 82, further comprising aligning the implant with at least one of the plurality of imaging markers.

Example 84. The method of any of examples 76-83, wherein the central body has a tapered profile, and the implant comprises a prosthetic heart valve.

Example 85. The method of any of examples 76-84, wherein the first end of the inflatable body is a proximal end and the second end of the inflatable body is a distal end.

Example 86. A method comprising:
dilating an implant utilizing an inflatable body having a central body pressed towards an inner surface of the implant, the inflatable body having a first end and a second end and a central body positioned between the first end and the second end, the central body including a plurality of segments each having a different profile than each other of the plurality of segments; and
aligning the implant with at least one of a plurality of imaging markers each configured to indicate a position of a respective one of the plurality of segments.

Example 87. The method of example 86, wherein the plurality of imaging markers comprise radiopaque markers.

Example 88. The method of example 86 or example 87, wherein the plurality of imaging markers are spaced from each other along a longitudinal axis of the inflatable body.

Example 89. The method of any of examples 86-88, wherein at least one of the plurality of segments has a profile angle that is at a different angle than a profile angle of another of the plurality of segments.

Example 90. The method of any of examples 86-88, wherein each of the plurality of segments has a profile angle that is at a same angle as each other of the plurality of segments.

Example 91. The method of any of examples 86-90, wherein each of the plurality of segments has a greatest diameter that is different than a greatest diameter of each other of the plurality of segments.

Example 92. The method of any of examples 86-91, wherein the central body has a tapered profile, and the implant comprises a prosthetic heart valve.

Example 93. The method of any of examples 86-92, wherein the central body has an angled profile.

Example 94. The method of any of examples 86-93, further comprising:
identifying at least one of the plurality of segments based on the plurality of imaging markers that have been imaged; and
selecting one of the plurality of segments for dilating the implant.

Example 95. The method of any of examples 86-94, wherein the first end of the inflatable body is a proximal end and the second end of the inflatable body is a distal end.

What is claimed is:

1. A dilation device for an implant, the dilation device comprising:
an inflatable body having a first end and a second end and a central body positioned between the first end and the second end and having a length, the central body configured to press an inner surface of the implant to dilate the implant and having a profile that decreases in diameter along the length of the central body from the first end to the second end.

2. The dilation device of claim 1, wherein the profile is a tapered profile.

3. The dilation device of claim 1, wherein the profile is an angled profile.

4. The dilation device of claim 1, wherein the central body includes a plurality of segments, at least one of the plurality of segments having a profile angle that is at a different angle than a profile angle of another of the plurality of segments.

5. The dilation device of claim 1, wherein the central body includes a plurality of segments, each of the plurality of segments having a profile angle that is at a same angle as each other of the plurality of segments.

6. The dilation device of claim 4, wherein each of the plurality of segments has a greatest diameter that is different than a greatest diameter of each other of the plurality of segments.

7. The dilation device of claim 1, wherein the central body includes a plurality of segments; and further comprising a plurality of imaging markers each configured to indicate a position of a respective one of the plurality of segments.

8. The dilation device of claim 7, wherein at least one of the plurality of imaging markers comprises radiopaque markers.

9. The dilation device of claim 7, wherein at least one of the plurality of imaging markers is positioned within an interior cavity of the inflatable body or is positioned on a wall of the inflatable body.

10. The dilation device of claim 1, wherein the central body includes a plurality of segments each having a constant diameter, at least one of the plurality of segments having a different diameter than another of the plurality of segments.

11. A dilation device for an implant, the dilation device comprising:
an inflatable body having a first end and a second end and a central body positioned between the first end and the second end and configured to press an inner surface of the implant to dilate the implant, the central body including a plurality of segments each having a different profile than each other of the plurality of segments; and
a plurality of imaging markers, wherein each imaging marker is configured to be indicative of a position of a segment of the plurality of segments.

12. The dilation device of claim 11, wherein the plurality of imaging markers comprises radiopaque markers.

13. The dilation device of claim 11, wherein the imaging markers are spaced from each other along a longitudinal axis of the inflatable body.

14. The dilation device of claim 11, wherein at least one of the plurality of segments has a profile angle that is at a different angle than a profile angle of another of the plurality of segments.

15. The dilation device of claim 11, wherein each of the plurality of segments has a profile angle that is at a same angle as each other of the plurality of segments.

16. An implant deployment system comprising:
an implant configured to be deployed to a deployment site within a subject and having an angled interior profile that faces an interior cavity of the implant; and
an inflatable body having a central body configured to be positioned within the interior cavity of the implant and configured to press towards the implant to dilate the implant, the central body having a profile that is angled to fit the angled interior profile of the implant.

17. The implant deployment system of claim 16, wherein the implant has a length and the central body has a length that is greater than the length of the implant.

18. The implant deployment system of claim 16, wherein the central body includes a plurality of segments each having a different profile than each other of the plurality of segments.

19. The implant deployment system of claim 16, wherein the inflatable body is configured to be in an undeployed configuration and is configured to be inflated to a deployed configuration.

20. The implant deployment system of claim 16, wherein the inflatable body is configured to not expand beyond a defined profile of the inflatable body upon inflation of the inflatable body.
